(19) United States
(12) Patent Application Publication
(10) Pub. No.: US 2018/0185661 A1
(43) Pub. Date: Jul. 5, 2018

(54) MEDICAL IMPLANT HOUSING HAVING ATTACHED SUTURE ANCHORS

(71) Applicant: InCube Labs, LLC, San Jose, CA (US)

(72) Inventors: Mir A. Imran, Los Altos Hills, CA (US); Robert Gaffney, San Jose, CA (US)

(21) Appl. No.: 15/854,518

(22) Filed: Dec. 26, 2017

Related U.S. Application Data
(60) Provisional application No. 62/441,053, filed on Dec. 30, 2016.

(51) Int. Cl. A61N 1/375; A61B 17/04; A61M 31/00; A61N 1/36; A61N 1/362

(52) U.S. Cl. CPC A61N 1/37518; A61B 17/0401; A61B 2017/00862; A61N 1/3605; A61N 1/362; A61M 31/002

(57) ABSTRACT

Embodiments provide implant housings (IH) having embedded suture anchors comprising a suture anchor loop (SAL) with an anchoring element each end. The SAL allows a suture to be passed through the loop to anchor the housing to selected tissue. Particular embodiments provide IHs having embedded sutures wherein the suture is embedded in a cured portion (CP) of the housing wall (HW). The suture may be part of a suture assembly having a first and second end portion (EP) and a mid-portion (MP) each of the EP's including an anchoring portion (AP). At least a portion of the HW comprises a cured portion (CP). The first and second SA EP's are embedded in the CP such that the MP forms a loop extending out from the HW outer surface. The embedded AE is configured such that a force for pulling the suture out of the CP exceeds a suture tensile strength.

### CROSS-REFERENCE TO RELATED APPLICATIONS

This application claims the benefit of priority of U.S. Provisional Application No. 62/441,053, filed Dec. 30, 2016, entitled Medical Implant Housing Having Attached Suture Anchors, the entire content of which is incorporated herein by reference for all purposes.

### BACKGROUND

### Field of the Invention

The present invention relates to medical implants. More particularly, embodiments of the present invention relate to medical implants having embedded sutures. Still more particularly, embodiments of the present invention relate to medical implant housings having embedded sutures in a portion of the housing.

Owing to the aging population, there is an increasing use of implanted devices for the treatment of a variety of conditions. In particular, there is an increasing use of implantable medical devices, such as implantable pacemakers which have housings that need to be sutured or otherwise anchored in place once implanted in the patient's body. Currently, such devices have suture holes or other spaces within the device housing to allow a suture to be inserted through the hole. However as the devices and housings get smaller, the hole occupies a much larger percentage of the device housing area reducing the internal volume of the housing for various components, making the housing more difficult to manufacture and also possibly mechanically weakening the housing. Also, the suture hole becomes a point of stress concentration both in the device housing and for the suture. What is needed is a means for attaching a suture to implanted device housing without occupying a space within the housing, such as hole for attaching the suture to the housing.

### BRIEF DESCRIPTION

Various embodiments of the invention provide medical implant housings having one or more embedded suture anchors. Many embodiments provide medical implant housings having embedded suture anchors comprising a suture anchor loop (herein suture loop) with an anchoring element on either end of the suture loop. The suture loop allows a suture to be passed through the loop so as to anchor the housing to selected tissue or a selected tissue site. Particular embodiments provide medical implant housings having embedded sutures wherein the suture is embedded in a portion of the housing wall, where that portion of the wall comprises a curable polymer such as epoxy or silicone. One embodiment provides a housing for a medical implant comprising a housing wall, and a suture assembly having a first end portion, a second end portion and a mid-portion each of the end portions including an anchoring portion. In preferred embodiments, the suture anchor is fabricated (e.g. by molding) as a single component including the mid-portion and end portions with anchoring portions. The housing wall defines an interior volume of the implant. The wall also includes an inner and outer surface and a wall thickness wherein at least a portion of the wall comprises a cured portion formed from a curable polymer material such as epoxy. The first and second end portions of the suture are embedded in the cured portion of the wall such that the mid-portion forms a loop extending out from the outer wall surface. The embedded anchoring element is configured such that a force for pulling the suture out of the cured portion exceeds a tensile strength of the suture typically, the portion comprising the mid-portion. According to various embodiments, the pull out force for the suture exceeds five pounds, more preferably seven pounds and still more preferably ten pounds. In various embodiments, the suture anchor can also include a re-enforcing element placed over the housing where the end portions exit the housing. The re-enforcing element serves to increase the pull out strength of the suture anchor and may be a circular or other like shape with a hole in the center for passage of the suture anchor.

The anchoring element can have a variety of shapes. According to various embodiments, suitable shapes for the anchoring element may include for example, spherical, semispherical, cylindrical, T-shaped and X-shaped. Also according to one or more embodiments, both ends of the suture anchor can be attached to the same anchoring element. In these and related embodiments, the anchoring element can have a cylindrical shape which is configured to have a longitudinal axis that is substantially parallel to a surface of the housing. In this case substantially parallel being an angle between the longitudinal axis and the surface of the housing that is equal or less than about 5°.

In particular embodiments, the suture anchor can be fabricated from sufficiently resilient materials such that the mid-portion stands erect and/or springs back to an erect position when depressed, for example, when pushed down by a surgical instrument. Further in specific embodiments, the suture anchor has sufficient spring force to spring back to its upright position when not only deflected but also if there are forces or conditions present for adhering it to a surface of the housing, such as those present if the suture anchor becomes wetted with blood or other fluid (e.g., saline) on the surface of the housing or nearby location. In use, such embodiments provide a suture anchor that is easier to both pass through and attach a suture to since the suture anchor is flexible enough to bend when the surgeon passes a needle through it but then flexes back to an upright position when bent or pushed against a surface wetted with blood or other fluid. In additional embodiments, the spring force of the suture anchor is also configured to be in range such that the suture anchor will spring back to its upright position after being depressed but at the same time is not so rigid that it would cause injury or irritation or even deformation to surrounding tissue when pressed against it as a result of movement of tissue or the housing in the body. In use such embodiments of the suture anchor allow the medical implant housing to be retained or tethered at a desired tissue location while easily floating or otherwise moving within the tissue site in response to applied forces from physiologic function (e.g., respiration or cardiac function) or patient movement so as to not irritate or interfere with organs or other tissue near or at the tissue site. For example, the housing could readily move or float so as to not impede blood vessels (e.g. arteries or veins) or lymphatic vessels or to impinge on a nerve.

Also, in related embodiments, to further facilitate maintenance of the suture anchor in an upright position when exposed to bodily fluids (e.g. blood, CSF, urine, intestinal fluids, etc.) or other fluid (e.g., saline), the suture anchor can be fabricated from hydrophobic materials and/or have a hydrophobic coatings, such as silicone or PTFE such that the suture anchor does not stick or adhere to the housing or tissue when either is wetted with blood or other fluids. In use, such embodiments allow the surgeon to more easily get a suture through the suture anchor since the suture anchor will remain in an erect position even after becoming wetted with tissue fluids such as blood which may cause the suture anchor to adhere to the housing surface or surrounding tissue.

In alternative or additional embodiments, the suture anchor can be fabricated from radiopaque and/or echogenic materials so that the suture anchors are easily visible using various imaging modalities (e.g., ultrasound, fluoroscopy and the like). In use such embodiments allow the doctor or other medical practitioner to determine if the housing is still attached to the desired anchor point or points in the patient (e.g., bone, vertebrae, cartilage, etc.).

In still other embodiments, the suture anchors can be configured to be selectively placed on the housing by the doctor and then fixed in place. In one or more embodiments, this can be accomplished by means of a slot or track within the surface of the housing which allows one or both ends of the suture anchors including the anchor element to be moved within and then be locked in place so that the surgeon can position the suture anchor where they want. In additional or alternative embodiments for achieving this, the coating or other layer above the implant housing can be configured to have anchoring wells or other attachment sites that are configured to receive the anchor element. The anchoring wells can be configured to fix or otherwise lock the anchor in place. In related embodiments, a curable adhesive, such as a curable epoxy, can be then placed over the anchor wells to adhere the anchor element in place.

In additional embodiments, the suture anchor may be coated with various functional coatings configured to perform various functions. Such functional coatings may include for example, an antibiotic, non-thrombogenic, or anti-cell adherence coating so as to reduce bacterial adhesion and/or prevent clots or tissue adherence to the suture anchor. The coating may be configured to elute one or more of an antibiotic, anti-thrombogenic or anti-cell adherence agent. According to particular embodiments, the coating may comprise a cyto-static agent such as Sirolimus so as to reduce cell adherence and/or proliferation on the suture anchor. Other cyto-static agents known in the art are also considered.

According to one or more embodiments, the anchoring elements can be embedded in an epoxy or other material used to coat the housing surface so that the suture loop protrudes from the coating. In an embodiment of a method for fabricating an implant housing having suture anchors, the anchor elements can be attached at the desired location on the housing, for example, using an adhesive, and the housing surface is then coated with the epoxy or other coating material such that the suture loop protrudes through the coating a selected amount. In preferred embodiments the housing surface is dip coated but other coating methods are also contemplated. As described herein, the height of the suture loop can be selected to allow a surgical needle and/or forceps (holding the needle) to be easily passed through the loop. Similarly, it may also be configured to allow forceps, micro-grabbers or like instruments or devices from a robotical surgical device to be used. In particular embodiments, the height of the suture loop above the coated surface of the housing can be in a range from about 0.25 to 1 inches.

Such embodiments of implanted housings having embedded suture anchors are particularly useful for a number of medical implants having a housing including for example, implanted cardiac devices such as cardiac pacemakers and defibrillators, implanted neuro-stimulators and implanted drug delivery pumps.

Further details of these and other embodiments and aspects of the invention are described more fully below, with reference to the attached drawing figures.

### BRIEF DESCRIPTION OF THE DRAWINGS

FIG. 1 is a lateral view showing an embodiment of a housing having a suture anchor.

FIG. 2 is a lateral view showing an embodiment of the suture anchor including a loop and anchoring elements.

FIG. 3 is a perspective view showing placement of the suture anchor on the corner of an implant housing.

FIGS. 4a, 4b, 4c and 4d are lateral views showing different embodiments of the suture anchor having different shaped anchoring elements.

FIG. 5 is a lateral view showing an embodiment of the implant housing having four suture anchors distributed around a perimeter of the housing wall.

FIG. 6 is a lateral view showing an embodiment of the suture anchor having a ringlet configuration.

FIGS. 7a, 7b, 7c and 7d are lateral views showing embodiments of the suture anchor including a strain gauge which may comprise piezo-electric fiber or other piezo-electric material. FIG. 7b illustrates an embodiment of a suture anchor having a coaxial orientation of its piezo electric fibers with respect to the central axis of the suture anchor; FIG. 7c shows an embodiment of a suture anchor having three piezo electric fibers distributed around the central axis of the suture anchor; and FIG. 7d shows an embodiment of a suture anchor having five piezo electric fibers distributed around the central piezoelectric fiber and which also may be configured as an energy harvesting mechanism.

### DETAILED DESCRIPTION OF THE INVENTION

Various embodiments of the invention provide medical implant housings having one or more embedded sutures anchors or other anchoring means. Many embodiments provide medical implant housings having embedded suture anchors comprising a suture anchor loop with an anchoring element on either end of the suture loop. The suture loop is configured (e.g. via length, shape etc.) to allow a suture to be passed through the loop so as to anchor the housing to selected tissue or a selected tissue site so as to secure the implant housing (e.g., a header or can used in an implantable cardiac pacemaker). Particular embodiments provide medical implant housings having embedded sutures wherein the suture is embedded in a portion of the housing wall which comprises a curable polymer such as epoxy or silicone. As used herein the term, “about” means within ±10% of a stated property, dimension or other value and, more preferably, ±5% of the stated value. Also as used herein the term “substantially” means within ±10% of a stated property or quality, more preferably, ±5% of the stated value. So, for example, a substantially symmetrical distribution of suture anchors around the housing wall perimeters means that the suture anchors are within 10% of having a symmetrical distribution and more preferably within 5%.

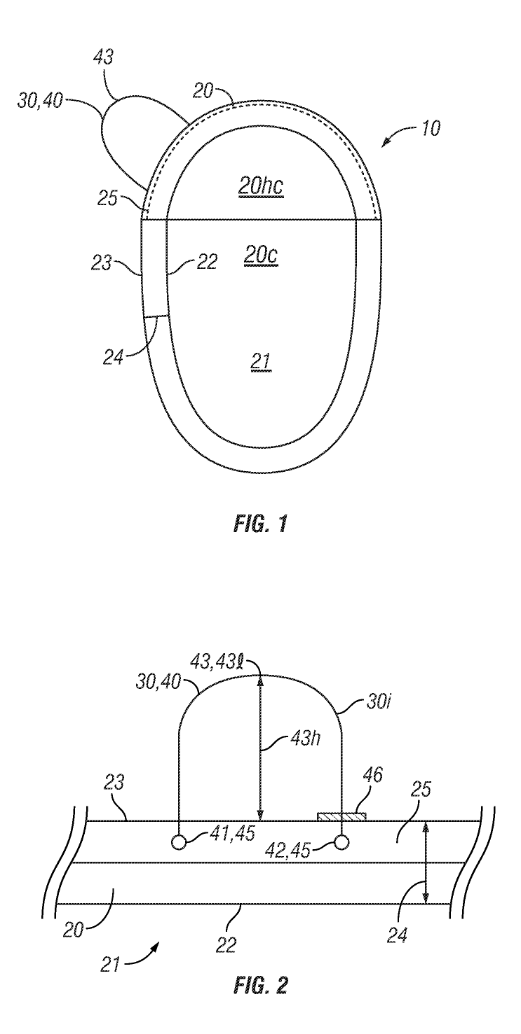

Referring now to FIGS. 1-6, an embodiment of a medical implant housing 10 having one or more suture anchors 30 may comprise a housing wall 20 and a suture anchor loop 40 (herein suture loop 40) having a first end portion 41, a second end portion 42 and a mid-portion 43. The medical implant housing may comprise one or both of a header or can be used, for example, with an implantable cardiac device or implantable neuro-stimulator. Each of the end portions 41 and 42 will typically include an anchoring element 45. The housing wall 20 defines an interior volume 21 of the implant housing. The wall 20 also includes an inner and outer surface 22 and 23 and a wall thickness 24. In many embodiments, at least a portion of the wall 20 comprises a layer 25 formed from a curable polymer material 26 such as epoxy, with other curable polymers also considered. For ease of discussion, layer 25 will now be described as cured portion 25, though it should be understood that non curable materials are also contemplated for layer 25. The first and second end portions 41 and 42 of the suture loop 40 will typically be embedded in the cured portion 26 of the wall 20 such that the mid-portion 43 forms a loop portion 431 extending out from the outer wall surface 23 through which a suture can be passed.

In preferred embodiments, the suture anchor 30 is fabricated as a single or unitary structure 30i including the mid-portion 43 and end portions 41 and 42 with their respective anchoring portions 45. The length, width and material properties of suture anchor 30 (e.g., bending modulus) can be selected to produce a loop portion 431 having a selected height and resiliency/stiffness such that the loop portion will resume an upright shape if deflected by an external force. Fabrication of a unitary suture anchor structure 30i can be done by molding or other process known in the polymer arts, e.g. extrusion. In use, embodiments of the invention providing a unitary suture anchor 30i structure may provide several advantages including: i) a suture anchor which has structure that results in increased tensile strength and pull out strength from the implant housing; ii) a suture anchor that has a structure which can easily be attached to a medical implant housing, (e.g., by having anchor elements that can easily be embedded in a cured portion or other coating applied to the housing surface); and iii) a suture anchor which has a structure that once embedded (or otherwise attached to the implant housing) produces a loop portion 431 with a selected shape and one that is sufficiently resilient to return to an erect position if deflected. In an exemplary embodiment of a method of using such a suture anchor 30 having a unitary structure 30i, the ends portions 41 and 42 including suture anchors 45 are embedded in coating 25 and then cured or otherwise adhered in place within or on coating 25 and/or housing surface 20. This results in a suture loop portion 431 which projects from housing wall 20 by a selected length and has a selected resiliency/stiffness to resume its original upright shape when deflected.

Anchoring element 45 is desirably shaped and configured so as to anchor the suture loop 40 to housing wall 20 and housing 10. In many embodiments, this is accomplished by embedding the anchoring elements in cured portion 25. It should be appreciated though that anchoring elements 45 may also be attached to housing wall 20 by other means including through the use of a separate adhesive, ultrasonic welding, solvent bonding, heat stacking and mechanical joints as well as combinations of one or more of these methods. In one or more embodiments, the anchoring element 45 can be embedded in cured portion 25 or otherwise attached to housing wall 20 such that a force for pulling the suture loop 40 out of the cured portion 25 (and/or wall 20) exceeds a tensile strength of the suture loop 40 itself, typically the tensile strength of the loop or mid-portion 43. According to various embodiments, the pull out force for the suture loop 40 exceeds five pounds, more preferably seven pounds and still more preferably ten pounds. In various embodiments, the suture loop 40 can include a re-enforcing element 46 placed over the housing wall 20 where the end portions 41 and 42 exit the housing. The re-enforcing element 46 serves to increase the pull out strength of the suture anchor 30 and may include a circular or other like shape with a hole in the center for passage of the suture anchor 30.

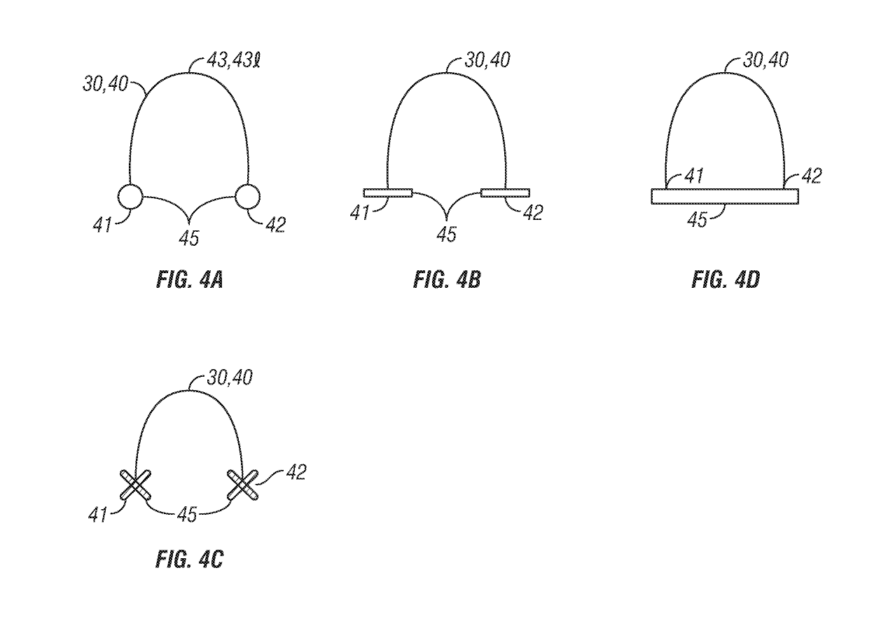

The anchoring element 45 can have a variety of shapes 45s. According to various embodiments suitable shapes 45s can include for example, spherical, semispherical, cylindrical, T-shapes and X-shapes. The embodiments shown in FIGS. 4a-4c, illustrate embodiments of spherical, T-shaped and X-shaped anchoring elements. Also according to one or more embodiments, both ends of the suture anchor 30 can be attached to the same anchoring element 45 as is shown in the embodiment of FIG. 4d. In these and related embodiments, the anchoring element 45 can have a cylindrical shape which is configured to have a longitudinal axis that is substantially parallel to a surface of the housing. In this case, substantially parallel being an angle between the longitudinal axis and the surface of the housing that is equal or less than about 5°. In particular embodiments, the anchoring elements 45 and other portions of suture loop 40 (e.g., end portions 41 and 42, mid-portions 43) may be annealed after being embedded into coating 25 so as to relieve any stresses imparted during attachment of suture loop 40 to or into coating 25 or other portions of housing wall 20.

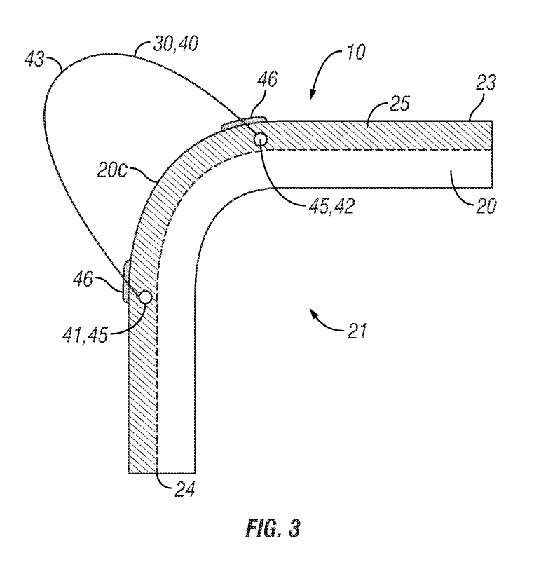

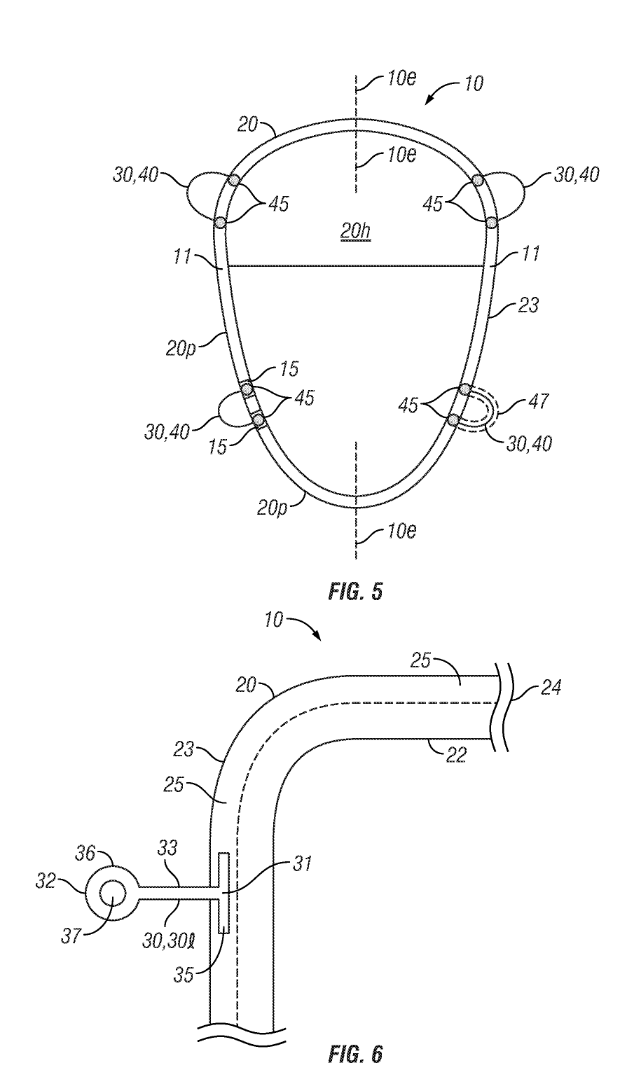

Suture anchors 30 may be placed in any number of locations on implant housing wall 20. In particular embodiments it may be positioned on one or more corners 20c of housing wall 20 as is shown in the embodiment of FIG. 3. In various embodiments, multiple suture anchors 30 may be positioned around housing wall 20 so as to anchor it at a desired location in the body (e.g. the pectoral region, spinal region lower back, etc.). In various embodiments, 2, 3, 4 or more suture anchors 30 may be positioned around housing wall 20. Further they may be positioned substantially symmetrically or asymmetrically around the perimeter 20p of housing wall 20. In the embodiments shown in FIG. 5, four suture anchors 30 are positioned around the perimeter of implant housing wall 20. Also as shown in FIG. 5, the suture anchors 30 may be substantially symmetrically positioned on header portion 20h or can portion 20c of housing 10 and housing wall 20. Depending upon the shape of housing 10, the number of suture anchors 30 selected and their position along the perimeter 20p of housing wall 20 can be configured so as to have a substantially equal distribution of stress on each suture anchor. In preferred embodiments, the number of suture anchors selected for such configurations can be 3 or 4. Also, they can be positioned substantially symmetrically along housing wall perimeter 20p. In embodiments of the invention having movable suture anchors 30, the surgeon can readily move and/or adjust the suture anchors 30 to their desired locations so as to obtain a substantially symmetrical or other distribution of suture anchors 30 for the particular implant site selected and their positions are selected to fix the suture anchors in place as is described herein. In the embodiment shown in FIG. 5, the suture anchors 30 can be moved around the surface of housing wall 20 by means of a slot positioned on a perimeter 20p of the housing wall 20 aligned with a length wise axis 101 of the housing 10. Other means for moving suture anchors around housing 10 are also contemplated, such as the use of anchor wells 15 described herein.

In particular embodiments, the suture anchor 30 can be fabricated from sufficiently resilient materials and have sufficient thickness such that the mid-portion 43 stands erect and/or springs back to its original erect position when depressed or otherwise deflected, for example, when pushed down by a surgical instrument or when temporarily pressed against tissue. Further, desirably suture anchor 30 has sufficient spring force to spring back to its upright position when not only deflected but also if it should become temporally adhered to housing wall 20 or other tissue surface through the action of blood or other tissue fluid (e.g. wetting or clotting), tissue adhesion or cellular deposition or other like process. In various embodiments, the amount of spring force of the mid-portion 43 (i.e., the force with which it springs back to an upright position when bent at a 90° angle) can be in the range of about 0.01 to 1 lbs, with specific embodiment of 0.05 to 1 lb, 0.1 to 1 lb, 0.1 to 0.5 lb, 0.2 to 1 lb and 0.2 to 0.5 lbs.

According to other embodiments, the spring force of the suture anchor 30 is also configured to be in a range such that the suture anchor 30 will spring back to its upright position after being depressed but at the same time, is not so rigid that it would cause injury or irritation or even deformation to surrounding tissue when pressed against it as a result of movement of tissue against the housing or movement of the housing 10 in the body (e.g. from patient movement either external or internal such as from breathing or the beating heart). In use, such embodiments of the suture anchor 30 allow the medical implant housing 10 to be retained or tethered at a desired tissue location/implant site while easily floating or otherwise moving within the body in response to applied forces from physiologic function (e.g., respiration, cardiac or digestive function) or patient movement so as to not irritate or interfere with organs, organ function or other tissue near or at the implant site. For example, the housing could readily move or float so as to not impede blood vessels (e.g. arteries or veins) or the lymphatic vessels and the movement of fluid though either or to impinge one or more nerves at or near the implant site. In these and related embodiments, the various embodiments, the amount of spring force of the mid-portion 43 (i.e., the force with which it springs back to an upright position when bent at a 90° angle) can be in the range of about 0.01 to 0.5 lbs, more preferably in the range of about 0.01 to 0.2 lbs and still more preferably in a range from about 0.01 to 0.1 lbs. In related embodiments suture loop 40 can have a combination of stiffness and flexibility such that the loop portion 43 will maintain its loop shape when it is bent 90° or more for example, by being placed closely against nearby tissue. In use, such embodiments allow implant housing 10, having suture anchors 30 and suture loop 40, to be implanted into a tight fitting implant site where there is little or no room for the suture loop 40 to fully extend to its normal loop shape, but instead, have the suture loop 40 deflect 90° or more so as to project upward out of the juncture between the implant housing wall 20 and adjacent tissue. This in turn allows the surgeon to readily access and suture to the suture loop 40 even when the implant housing 10 is implanted in tight fitting spaces. In these and related embodiments the bending stiffness/spring force of the suture loop 40 can be in a range of about 0.005 to 0.5 lbs, more preferably in the range of about 0.005 to 0.2 lbs, and still more preferably in a range from about 0.01 to 0.1 lbs.

Suitable materials for suture loop 40 may correspond to one or more of NYLON or other Polyamide, polypropylene, silk, polyester and any copolymers thereof. These or other materials may be formed into either monofilament or a braided design. The diameter or gauge of the suture can range from 1 to 4, with the particular diameter selected for one or more of the implant sites' desired strength and stiffness of the suture, including bending stiffness/flexibility as measured by bending modulus. The suture may also be coated with one or more biocompatible coatings known in the art. These coatings may also be functionalized as is described herein.

Also according to particular embodiments, suture loop 40 can be fabricated from hydrophobic materials and/or have hydrophobic coatings, such as silicone or PTFE which are selected and configured such that the suture loop does not stick or adhere to the housing or tissue when either it is wetted with blood or other bodily fluid blood (e.g., CSF, urine, intestinal fluids, etc.) or other fluid (e.g., saline). In use, such embodiments allow the surgeon to more easily get a suture through the suture loop 40 since the suture loop will remain in erect position even when pushed down. In alternative or additional embodiments, the suture loop 40 can be fabricated from radiopaque and/or echogenic materials so that they are visible using various imaging modalities (e.g. fluoroscopy, ultrasound, etc.). Further in specific embodiments, the suture loop 40 has sufficient spring force to spring back to its upright position when not only deflected but also if there are forces or conditions present for adhering it to a surface of the housing, such as those present if the suture loop 40 becomes wetted with blood or other fluid (e.g., saline) on the surface of the housing or nearby location. In particular embodiments, this spring force may in the range of 0.2 to 11b. In use, such embodiments provide a suture loop 40 that is easier to both pass through and attach a suture to since the suture loop 40 is flexible enough to bend when the surgeon passes a needle through it, but then flexes back to an upright position when bent or pushed against a surface wetted with blood or other fluid including housing wall 20 itself.

In alternative or additional embodiments, the suture loop 40 of suture anchor 30 can be configured to be self-synching such that once the doctor passes a suture through the suture loop 40, it can be pulled taught so that the external suture is pulled adjacent to the housing 10. This approach can be accomplished through a variety of means including the use of various one way ratchets and/or clamp mechanisms known in the art.

In other embodiments, suture anchor 30 may have other shapes besides a loop. For example, according to an embodiment shown in FIG. 6, suture anchor 30 may have a substantially linear shape 30l comprising a substantially straight portion 33 having at one end 31, an anchoring element 35 embedded a selected depth in coating 25 (or other portion of wall 20) and at the other end 32 an eyelet element 36 having an opening 37 sized and configured to allow the passage of a suture through. Anchoring element 35 may have any of the shapes described herein for anchor element 45, for example, T-shaped as shown in the embodiment of FIG. 5. Also the straight portion 33 may extend out of coating 25 the same amounts as described for suture loop 40. In these and related embodiments of a linear-shaped suture anchor 30, the suture anchor may be configured such that straight portion 33 has similar flexibilities (e.g. spring force) such as that described for suture loop 40. For example, it may be easily bent 90° over by the application of force from forceps or being pushed against other tissue but then springs back to its original position once the force is removed. In other embodiments, it may be substantially rigid so that it does not appreciably bend or flex with the application of such forces. The flexibility of the linear suture anchor 30l can be controlled by selection of one or more of the materials, thickness and penetration depth into coating 25 of the anchoring element 35.

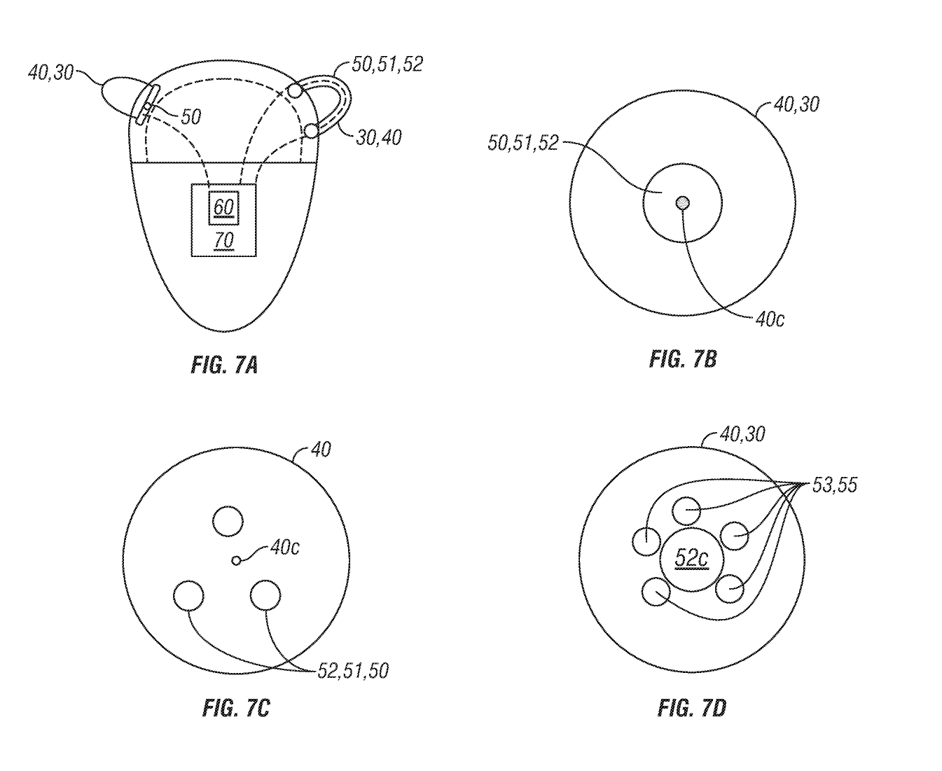

Referring now to FIGS. 7a-7d, in still other alternative embodiments, suture loop 40 may include or be operatively coupled to a strain gauge 50 to let the doctor know how much tension he/she is putting the suture loop under when they tie a suture to it The strain gauge 50 can also be configured to be coupled to a microprocessor 60 associated with electronic circuitry 70 contained in housing 10 and may be further configured to provide an input to the microprocessor or like device of the stress on the various suture anchors 30 over the life of the implant. The microprocessor may include or be coupled to an transmission device such as RF transmitter for transmitting signals indicative of the stress on suture anchors 30. In use, such embodiments can be used to monitor for one or more of the following: i) any breaks in the suture anchors or attached sutures, 2) changes in stress indicative of a likely suture or suture anchor failure; or 3) any unusual in vivo stresses that the implant housing is subjected to, for example, those indicative of an adverse condition such as unusual tissue growth, infection or thrombus formation on or near the implant housing 10. In particular embodiments, the strain gauge 50 and microprocessor can be configured to allow the surgeon to put equal amounts of tension on all the suture loops 40 for implant housing 10. In use, such embodiments reduce the risk of tearing of the housing from a particular tissue site due to unequal tension on the suture loops. In various embodiments, the strain gauge may comprise one or more piezo electric materials 51, including for example, piezo electric fibers 52 configured to generate a voltage proportional to the amount of tension (or other force) the suture anchor 40 is put under. In particular embodiments, all or a portion of the suture anchor loop 40 including one or more of end portions 41 and 42 (including anchors 45) and mid-portion 43 may include piezo electric fibers or other piezo electric material. In these and related embodiments piezo electric fiber 52 may have coaxial configuration with respect to central axis 40c of the suture loop 40 as shown in FIG. 7b or a non-axial configuration where the piezo-electric fibers are distributed around the central axis of the suture anchor as shown in FIG. 7c. Such coaxial or multiaxial configurations can be produced using co-extrusion methods known in the art where a polymer comprising anchor loop 40 is co-extruded over fibers 52. In yet another embodiment shown in FIG. 7d, the piezoelectric fibers may comprise a central fiber 52c with five or more other fibers 53 distributed around the central fiber 52c. Embodiments of this and related configurations of piezoelectric fibers 52 may also be configured to function as an energy harvesting mechanism 55 for powering electronics 70. Further description of such piezo electric fibers and their configuration may be found in U.S. application Ser. Nos. 12/561,159 and 12/556,524 which are incorporated by reference herein for all purposes

In still other embodiments, the suture loop 40 can be configured to be selectively placed on housing 10 by the doctor or other medical practitioner and then fixed in place. In one or more embodiments, this can be accomplished by means of a slot or track 11 on or within the outer surface 22 of the housing 10 shaped or otherwise configured to allow one or both ends 41 or 42 of the suture loop 40 including the anchoring element 45 to be moved within and then be locked in place so that the surgeon can position the suture loop 40 where they want. An example of such a slot 11 is shown in FIG. 5 where the slot can be placed along a perimeter 20p of the housing wall. In additional or alternative embodiments for achieving this, the coating or other layer 25 above the implant housing 10 can be configured to have anchoring wells 15 or other attachment sites 15 that are configured to receive the anchoring element 45. The anchoring wells 15 can be configured to fix or otherwise lock the anchoring element 45 in place. In related embodiments, a curable adhesive, such as a curable epoxy that can then be placed over and/or injected into the anchor wells 15 to adhere the anchoring element 45 in place.

In additional embodiments, the suture loop 40 may be coated with various functionalized coatings 47 (also known as functional coatings 47) configured to perform various functions. Such functional coatings 47 may include for example, an antibiotic, non-thrombogenic or anti-cell adherence coating so as to reduce bacterial adhesion and/or prevent clots or tissue adherence to the suture anchor. The coating may be configured to elute one or more of an antibiotic, anti-thrombogenic or anti-cell adherence agents. According to particular embodiments, the coating may comprise a cyto-static agent such as Sirolimus and/or its analogues so as to reduce cell adherence and/or proliferation on the suture anchor. Other cyto-static agents known in the art are also considered.

According to one or more embodiments, the anchoring elements 45 can be embedded in an epoxy or other material used to coat the housing outer surface 23 so that the suture loop 40 protrudes through the layer of coating 25 a selected amount. The anchoring element 45 can be embedded at varying depths within the coating for example, in the range of about 5 mm to about 30 mm, with specific embodiments of 10, 15, 20, 25 mm. The depth of embedment can be selected depending upon one or more factors including the thickness of the epoxy coating, shape of the anchoring element, desired pull out force and desired deflection force of the loop portion 431. Other coatings besides epoxy are also contemplated, such as polyurethane, silicone and PTFE and respective copolymers.

In an embodiment of a method for fabricating an implant housing having a suture anchor 30, the anchoring elements 45 can be attached at the desired location on housing wall 20, for example, using an adhesive, and the housing surface 23 is then coated with the epoxy or other coating material such that the loop portion 43 protrudes through the coating 25 a selected amount. In preferred embodiments, the housing surface is dip coated but other coating methods are also contemplated. As described herein, the height of the suture loop can be selected to allow a surgical needle and/or forceps (holding the needle) to be easily passed through the loop. Similarly, the suture loop and/or suture loop height may also be configured to allow forceps, micro-grabbers or like instruments from a robotical surgical device to be used to pass sutures through the loop or otherwise manipulate the loop. In particular embodiments, the height 43h of the suture loop 43 above the coated surface 23 of the housing 10 can be in a range from about 0.1 to 1 inches, with specific embodiments of 0.2, 0.25, 0.3, 0.5, 0.75 and 0.9 inches. Different heights being selected for advantageous positioning of device at a particular tissue site, e.g., the chest, torso, etc.

### Applicability of Embodiments of the Suture Anchors to Various Medical Implants

According to various embodiments, embodiments of the suture anchors 30 described herein can be configured to be utilized with any number of medical implants in the body including, for example, implantable pacemakers, defibrillators or other implantable cardiac devices (e.g., pacemakers and defibrillators), implantable neuro-stimulators including those used for brain, spinal, gastric or urinary stimulation; implanted drug pumps including, for example, implantable insulin pumps; implantable cochlear devices or other implanted hearing aid; and various implantable sensor devices. Further the embodiments of the suture anchors can be configured to be used for implants in any number of locations in the body, including for example, the spinal area, pectoral area, gastro-intestinal area, cranial area or intraventricular area; urinary tract area; or arm or leg area. Adjustments for using suture anchors 30 in one or more of these areas can be made in one or more of the following: i) the suture anchor materials selection to control for stiffness and biocompatibility at the particular site; ii) suture anchor shape (e.g. the shape or size or the loop portion 431); or iii) dimension of the suture anchor (e.g., length, width or other dimension of the suture anchor). For example, for spinal placement, stronger materials may be used for the suture anchor to assure fixation to a bone or nearby location. Also, for placement of the device in a blood contacting environment, the suture anchors can be fabricated from non-thrombogenic materials and/or include non-thrombogenic coating.

### CONCLUSION

The foregoing description of various embodiments of the invention has been presented for purposes of illustration and description. It is not intended to limit the invention to the precise forms disclosed. Many modifications, variations and refinements will be apparent to practitioners skilled in the art. For example, embodiments of the housing and suture loops can be adapted for use in various pediatric and neonatal applications (e.g. by being made smaller and/or with changes in shape) as well as any number of veterinary applications including, for example, various canine, feline, bovine and equine applications.

Elements, characteristics, or acts from one embodiment can be readily recombined or substituted with one or more elements, characteristics or acts from other embodiments to form numerous additional embodiments within the scope of the invention. Moreover, elements, characteristics or acts or the like that are shown or described as being combined with other elements, characteristics or acts can, in various embodiments, exist as stand-alone elements, characteristics or acts. Further still, embodiments of the invention specifically contemplate the specific exclusion (e.g., a negative recitation) of any element, characteristic or act or the like that is positively recited in any embodiment. Hence, the scope of the present invention is not limited to the specifics of the described embodiments, but is instead limited solely by the appended claims.

What is claimed is:

1. A housing for a medical implant, the housing comprising:
a wall defining an interior volume, the wall including an inner wall surface and an outer wall surface, at least a portion of the wall comprising a cured portion formed from a curable polymer material; and
a suture anchor having a first end portion, a second end portion and a mid-portion, the first end portion and the second end portion each having an anchoring element, the anchoring element of the first end portion and the anchoring element of the second end portion being embedded in the cured portion of the wall, the mid-portion forming a loop extending out from the outer wall surface, wherein the embedded anchoring elements of the first end portion and the second end portion of the suture anchor are configured such that a force for pulling the suture anchor out of the cured portion of the wall exceeds a tensile strength of the mid-portion.

2. The housing of claim 1, wherein the suture anchor has an integral structure.

3. The housing of claim 1, wherein the suture anchor includes a strain gauge.

4. The housing of claim 3, wherein the strain gauge comprises a piezo electric material or a piezo electric fiber.

5. The housing of claim 1, wherein the anchoring elements of the first end portion and the second end portion are connected.

6. The housing of claim 1, wherein the anchoring elements of the first end portion and the second end portion are integral.

7. The housing of claim 6, wherein the integral anchoring elements have a linear shape or a cylindrical shape.

8. The housing of claim 1, wherein the anchoring element has a T-shape.

9. The housing of claim 1, wherein the anchoring element has a disc shape.

10. The housing of claim 1, wherein the anchoring element has a spherical shape.

11. The housing of claim 1, wherein at least a portion of the embedded anchoring elements of the first end portion or the second end portion of the suture anchor have a textured surface configured to mechanically bond with the curable polymer material so as to increase the pull out force of either end portion.

12. The housing of claim 1, wherein the end portions have a pull out strength of at least five pounds.

13. The housing of claim 12, wherein the end portions have a pull out strength of at least seven pounds.

14. The housing of claim 13, wherein the end portions have a pull out strength of at least ten pounds.

15. The housing of claim 1, wherein the housing has at least one corner portion and the end portions are positioned on either side of the at least one corner portion.

16. The housing of claim 1, wherein the suture anchor comprises a resilient material configured such that the mid-portion of the suture anchor stands substantially upright above the housing once embedded in the cured portion.

17. The housing of claim 16, wherein the mid-portion of the suture anchor has sufficient spring force once deflected to overcome adhesive forces between the suture anchor and the housing in the presence of bodily or other fluid or between the suture anchor and tissue and return to its upright position.

18. The housing of claim 17, wherein the spring force is in a range from about 0.1 to 0.51b.

19. The housing of claim 1, wherein the anchoring elements are stress relieved or annealed after placement.

20. The housing of claim 1, wherein the anchoring elements are attached to an uncured portion of the housing wall.

21. The housing of claim 1, further comprising at least one re-enforcing element positioned over a portion of the housing wall where the mid-portion of the suture anchor emerges.

22. The housing of claim 1, wherein at least a portion of the suture anchor includes a functionalized coating.

23. The housing of claim 1, wherein the functionalized coating comprises at least one of a non-thrombogenic coating, an antibiotic or a cyto-static agent.

24. The housing of claim 1, wherein the housing is a housing for a cardiac device.

25. The housing of claim 24, wherein the cardiac device is cardiac pacemaker.

26. The housing of claim 1, wherein the housing is a housing for a neuro-stimulator.

27. The housing of claim 1, wherein the housing is a housing for an implantable pump.

28. A housing for a medical implant, the housing comprising:
a wall defining an interior volume, the wall including an inner wall surface and an outer wall surface, at least a portion of the wall comprising a cured portion formed from a curable material; and
a suture anchor having a first end portion, a second end portion and a mid-portion, the first end portion and the second end portion each having an anchoring element, the anchoring element of the first end portion and the anchoring element of the second end portion being embedded in the cured portion of the wall, the mid-portion forming a loop extending out from the outer wall surface, wherein the embedded anchoring elements of the first end portion and the second end portion of the suture anchor are configured such that a force for pulling the suture anchor out of the cured portion of the wall exceeds about one pound.

29. The housing of claim 28, wherein the force for pulling the suture anchor out of the cured partition exceeds about two pounds.

30. The housing of claim 28, wherein the force for pulling the suture anchor out of the cured partition exceeds about three pounds.

31. The housing of claim 28, wherein the suture anchor comprises a resilient material configured such that the mid-portion of the suture anchor stands substantially upright above the housing once embedded in the cured portion.

32. The housing of claim 31, wherein the mid-portion of the suture anchor has sufficient spring force once deflected to overcome adhesive forces between the suture anchor and the housing in the presence of bodily or other fluid or between the suture anchor and tissue and return to its upright position.

33. The housing of claim 32, wherein the spring force is in a range from about 0.1 to 0.51b.

34. A housing for a medical implant, the housing comprising:
a wall defining an interior volume, the wall including an inner wall surface and an outer wall surface, at least a portion of the wall comprising a cured portion formed from a curable material; and
a suture anchor having a first end portion, a second end portion and a mid-portion, the first end portion and the second end portion each having an anchoring element, the anchoring element of the first end portion and the anchoring element of the second end portion being embedded in the cured portion of the wall, the mid-portion forming a loop extending out from the outer wall surface, the suture anchor comprising a resilient material configured such that the mid-portion of the suture anchor stands substantially upright above the housing once embedded in the cured portion and wherein the mid-portion of the suture anchor has sufficient spring force once deflected to overcome adhesive forces between the suture anchor and the housing in the presence of bodily or other fluid or between the suture anchor and tissue and return to its upright position.
